(19) United States
(12) Patent Application Publication
(10) Pub. No.: US 2018/0119090 A1
(43) Pub. Date: May 3, 2018

(54) PROCESS FOR INCREASED YEAST BIOMASS

(71) Applicant: Research Foundation of the City University of New York, New York, NY (US)

(72) Inventors: Peter Lipke, Brooklyn, NY (US); Uthama Edupuganti, Jersey City, NJ (US)

(21) Appl. No.: 15/856,667

(22) Filed: Dec. 28, 2017

Related U.S. Application Data
(63) Continuation-in-part of application No. 15/023,047, filed on Mar. 18, 2016.
(60) Provisional application No. 61/879,761, filed on Sep. 19, 2013.

(51) Int. Cl. C12N 1/38; C12N 1/16; C12N 1/32

(52) U.S. Cl. CPC C12N 1/38; C12N 1/32; C12N 1/16

(57) ABSTRACT

The present invention relates to a process for enhancing the growth and increasing the biomass of yeast cultures. The addition of ethanol during log growth phase increases the yield of yeast biomass and products purified from the biomass.

### CROSS-REFERENCE TO RELATED APPLICATIONS

This application claims priority to and is a continuation-in-part of U.S. patent application Ser. No. 15/023,047 (filed Mar. 18, 2016) which is a national stage entry of PCT/US2014/056276 (filed Sep. 18, 2014) which claims priority to U.S. Provisional Patent application 61/789,761 (filed Sep. 19, 2013), the entirety of which are incorporated herein by reference.

### BACKGROUND OF THE INVENTION

The present invention relates to the field of biotechnology and yeast biomass. Yeast biomass has many applications. In the food industry it is seen as an excellent source of protein, nucleic acids and vitamins and useful to make bread, wine and beer. In non-food industries, such as the biofuel industry, it is used to produce ethanol. It has also been developed for human and veterinary medicine for the production of antibiotics, useful proteins and β-glucans. β-glucan is a biological immunomodulator such that it has the ability to prime and activate the immune system. Increasing the yield of yeast biomass has always been a challenge, therefore new methods and procedures are being developed to do so.

Some conventional processes utilize an admixture of ethanol and fermentable carbon but such processes are silent concerning the amounts of ethanol and fermentable carbon added and the timing of such additions, because these were not monitored in the process. Conventionally, users assume that production of ethanol lowers biomass and ethanol is often added to cultures to simulate this effect. The discussion above is merely provided for general background information and is not intended to be used as an aid in determining the scope of the claimed subject matter.

### BRIEF DESCRIPTION OF THE INVENTION

The present invention relates to a process for enhancing the growth and increasing the biomass of yeast cultures. The addition of ethanol during log growth phase increases the yield of yeast biomass and products purified from the biomass.

In a first embodiment, a process for increasing yeast biomass is provided. The process comprises culturing yeast cells in a growth medium comprising a fermentable carbon source; adding ethanol before cessation of growth; and permitting the yeast cells to continue to grow after the step of adding ethanol.

In a second embodiment, a process for increasing yeast biomass is provided. The process comprising sequentially: adding yeast cells to a growth medium comprising a fermentable carbon source; culturing the yeast cells in the growth medium for a least one hour; adding ethanol, after the at least one hour, to the growth medium such that the ethanol has a concentration of between 0.5 volume percent and 4.0 volume percent, wherein the adding ethanol occurs within ten hours of the adding yeast cells; and permitting the yeast cells to continue to grow after the step of adding ethanol.

This brief description of the invention is intended only to provide a brief overview of subject matter disclosed herein according to one or more illustrative embodiments, and does not serve as a guide to interpreting the claims or to define or limit the scope of the invention, which is defined only by the appended claims. This brief description is provided to introduce an illustrative selection of concepts in a simplified form that are further described below in the detailed description. This brief description is not intended to identify key features or essential features of the claimed subject matter, nor is it intended to be used as an aid in determining the scope of the claimed subject matter. The claimed subject matter is not limited to implementations that solve any or all disadvantages noted in the background.

### BRIEF DESCRIPTION OF THE DRAWINGS

So that the manner in which the features of the invention can be understood, a detailed description of the invention may be had by reference to certain embodiments, some of which are illustrated in the accompanying drawings. It is to be noted, however, that the drawings illustrate only certain embodiments of this invention and are therefore not to be considered limiting of its scope, for the scope of the invention encompasses other equally effective embodiments. The drawings are not necessarily to scale, emphasis generally being placed upon illustrating the features of certain embodiments of the invention. In the drawings, like numerals are used to indicate like parts throughout the various views. Thus, for further understanding of the invention, reference can be made to the following detailed description, read in connection with the drawings in which:

FIG. 1 shows growth curves of S. cerevisiae in fermentor and illustrates the effect of ethanol on increased biomass and growth rate;

FIG. 2 shows a graph of ethanol-enhanced growth curves using media with 0.12% fermentable C source (1×CMF) and 0.24% fermentable C source (2×CMF) media that illustrates the synergy of ethanol with CMF: growth on CMF+2% ethanol is greater than the sum of either C source alone;

FIG. 3A, FIG. 3B and FIG. 3C and FIG. 3D show graphs of ethanol enhanced growth of various yeast strains; and

FIG. 4 shows a graph of glucan yield in yeast biomass that figure illustrates that the CMF+ethanol medium results in 2-3-fold yield increase in production of glucan, a yeast natural product.

### DETAILED DESCRIPTION OF THE INVENTION

Efficient growth of yeast biomass requires the coordination of nutrient assimilation, energy generation, biosynthesis and cell division. Once nutrients are depleted to the point of reducing biosynthetic activity, yeast responds by decreasing their growth rate. Along these lines, a limitation to increasing yeast biomass involves carbon source preference. Carbon sources such as glucose and fructose are metabolized rapidly as the preferred source of energy, and yeast enter stationary phase soon after utilization of these sugars, which limits biomass yield and the amount of yeast-derived natural products. This cessation of growth has been attributed to a metabolic stress that results from nutrient depletion, catabolite repression, and/or oxidative stress (De Deken, J. Gen. Microbiol., 44, 149-156, 1966). The use of fed-batch methods in which the amount of C source is restricted only partially alleviates the problem. (Rocío Gómez-Pastor, Roberto Pérez-Torrado, Elena Garre and Emilia Matallana (2011)). Recent Advances in Yeast Biomass Production, Biomass—Detection, Production and Usage, Dr. Darko Matovic (Ed.), ISBN: 978-953-307-492-4, InTech; and Demirci et al. J. Agric. Food Chem. 47:2496-2500 (1999)).

The current invention addresses this problem with the addition of ethanol, a non-fermentable carbon source, during log phase growth. Addition of ethanol to yeast cultures facilitates better growth of any yeast strain grown on one or more carbon sources. In the presence of added ethanol, yeast continues to grow and metabolic consequences of carbon limitation appear to be absent. This result indicates that the yeast grows stress-free while simultaneously utilizing both fermentable and non-fermentable carbon sources in the media. The ethanol is added during log phase growth, before the growth rate begins to decrease. In one embodiment, the ethanol is added after fresh yeast cells have been cultured for at least one hour but within ten hours of starting the culture. The ethanol is kept at a concentration of between 0.5% (vol) and 4.0% (vol) in the growth medium. In one embodiment, the ethanol is between 1.0% (vol) and 3.0% (vol). The fermentable carbon source is between 0.05% (wt.) and 0.5% (wt) in the growth medium. The process generally occurs at a temperature between 24° C. and 40° C. The added ethanol may be a pure solution of aqueous ethanol with no additional additives.

In general, increasing the yield of yeast biomass involves adding ethanol to an exponentially growing culture of yeast, such as Saccharomyces cerevisiae, in log phase under carbon-limited growth conditions. Seemingly, the ethanol is utilized as a non-fermentable carbon source in addition to the fermentable carbon source (glucose, sucrose, etc.) existing in the media. The fermentable carbon source generates ethanol throughout the growth period adding a metabolic and cellular stress. The addition of ethanol in early log phase results in up-regulation of anabolic pathways that utilize this non-fermentable carbon source, and thereby reduces the stress from the resulting products of fermentation. Thus, the yeast cells become competent to metabolize the non-fermentable and fermentable carbon sources simultaneously leading to increased biomass production.

### Example 1

A protocol for Cane Molasses Base Media (CMB) preparation was adapted from Demirci A et. al, (J Agric Food Chem. 1999 June; 47(6):2496-500). A liter of CMB was prepared based on the following composition:

| | | |
|---|---|---|
| CMF* | 7.5 mL | per liter |
| Na₂SO₄ | 1.13 g | per liter |
| CaCl₂•2H₂O | 0.14 g | per liter |
| MgCl₂•6H₂O | 0.94 g | per liter |
| KH₂PO₄ | 3.0 g | per liter |
| D-biotin | 0.4 mg | per liter |
| Trace element solution | 1 mL | per liter |

The Trace element solution was prepared as follows:

| | | |
|---|---|---|
| MgSO₄•7H₂O | 3.0 g | per liter |
| MnSO₄•H₂O | 0.5 g | per liter |
| NaCl | 1.0 g | per liter |
| FeSO₄•7H₂O | 0.1 g | per liter |
| CoSO₄•5H₂O | 0.18 g | per liter |
| CaCl₂•2H₂O | 0.08 g | per liter |
| ZnSO₄•7H₂O | 0.1 g | per liter |
| CuSO₄•5H₂O | 0.01 g | per liter |
| Al₂(SO₄)₃•nH₂O | 0.01 g | per liter |
| H₃BO₃ | 0.01 g | per liter |
| Na₂MoO₄•2H₂O | 0.01 g | per liter |

The solution was filter sterilized after preparation.

*Cane Molasses Feeding media (CMF):

| | | |
|---|---|---|
| Cane Molasses | 530 g | per liter |
| Urea | 21.7 g | per liter |
| CaCl₂•2H₂O | 0.14 g | per liter |
| NH₄H₂PO₄ | 4.0 g | per liter |
| MgCl₂•6H₂O | 0.94 g | per liter |
| KH₂PO₄ | 3 g | per liter |
| Trace Element solution | 1 mL | per liter |

*Urea & KH₂PO₄ filter sterilized & added to media after heat sterilization

In the following experiments, the concentration of CMF added to CMB media ranged from 1-4 times (1×-4×) the original, published concentration of 7.5 ml/Liter of medium Demirci A et. al, (J Agric Food Chem. 1999 June; 47(6):2496-500).

To grow in a shake flask, a starter culture was set up in a 250 ml flask with 50 ml of CMB+CMF media. The culture was grown overnight at 30° C. and 170 rpm in a shaker incubator. The experimental cultures in CMB with added CMF at 1× (7.5 ml/L), 2× (15 ml/L), 3× (22.5 ml/L), or 4×CMF (30 ml/L) were inoculated by diluting the starter culture 1:5 or 1:10 to get a starting OD600 of 0.3 or 0.150, respectively. The cultures continued incubation at 30° C. and 170 rpm in a shaker incubator. When the OD600 of the culture reached 0.45 to 0.8, which is usually around two generations (about 5 hours) from inoculation, ethanol was added to a concentration of 2 volume percent. In one embodiment growth is allowed to occur for at least four hours before ethanol is added. The culture was grown and additional OD's were taken periodically for a maximum of 120 hours. The results show that cultures with added ethanol have double the biomass of cultures without ethanol. This effect is apparent after 24 hours of culture in CMB+1×CMF medium, and at 40-120 hours in cultures with 2×, 3×, or 4×CMF.

### Example 2

For batch fermentation, a Sartorius BIOSTAT® Aplus bench top reactor equipped with Airflow gassing system, efficient agitation system, pH control and temp control was employed. The 1 L working volume vessel was equipped with air, alkali, acid and medium inlets ports. A temperature of 30° C., aeration of 1.3 l/min and agitation of 400 rpm was maintained throughout the experiment, as was addition of CMF at a rate of 0.1875% volume/volume per 24 hours. The cultures were inoculated from starter cultures as described in Example 1. Ethanol 2% (vol) was added at OD_{600 nm} between 0.45 and 0.8, about 5 hours after inoculation. The samples were taken and analyzed for cell density to establish growth curve of a Saccharomyces cerevisiae strain. Samples of the cultures were taken at 12, 24 and 32 hours to analyze the total biomass yield and glucan contents.

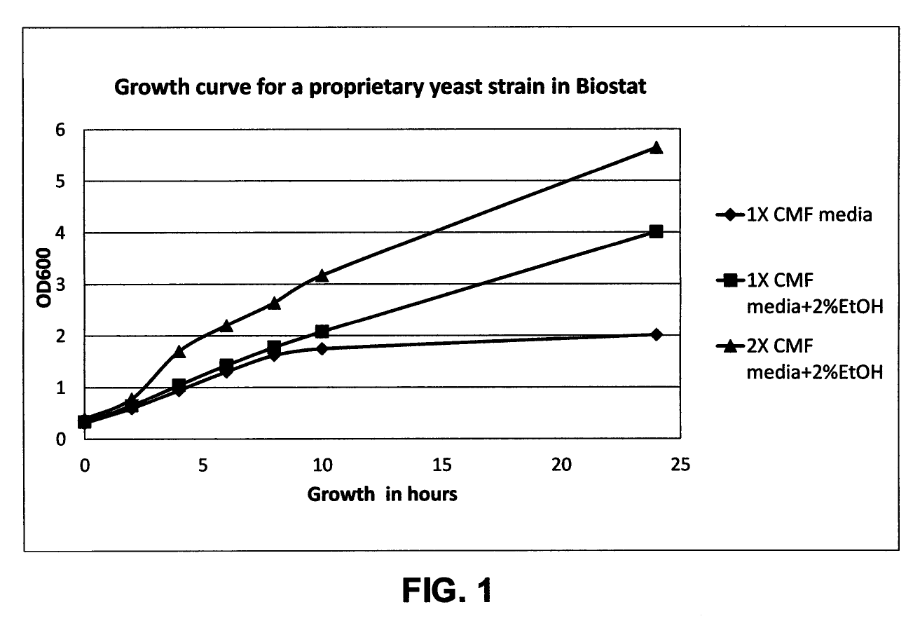

As shown in FIG. 1, S. cerevisiae grown in a fermentor in CMB-CMF medium supplemented with 2% ethanol shows increased growth rate and prolonged growth. In particular, 1×-CMB-CMF+2% ethanol media shows improved growth over media alone (control), while 2×-CMB-CMF+2% ethanol media provides even better growth rates and prolonged growth over the control.

### Example 3

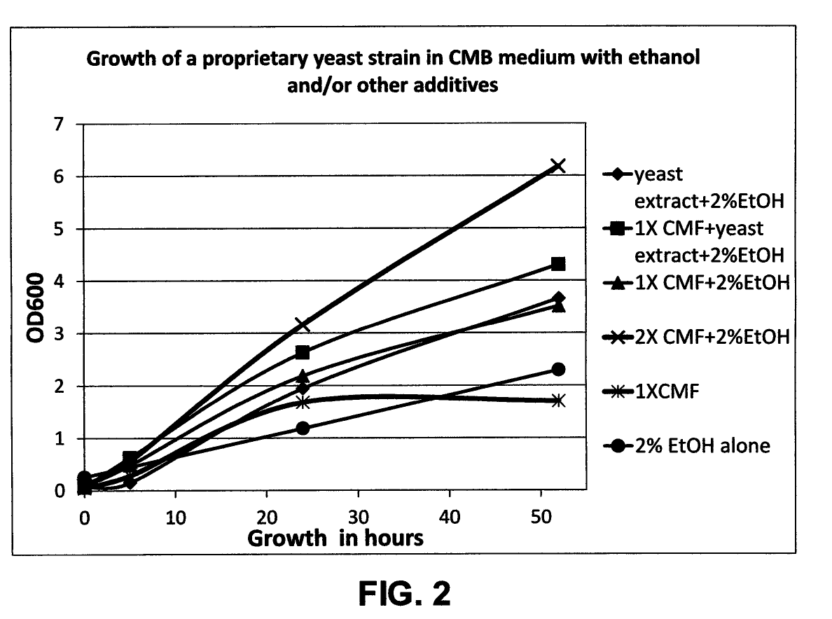

One way to optimize growth conditions and biomass volume is by varying the concentration of fermentable carbon sources (1-4× about 0.06% to 0.24% glucose+fructose). Ethanol-induced biomass yield and growth enhancement was also evident using any of a number of types of growth media, as long as fermentable Carbon sources are kept below 0.5% and ethanol kept above 0.5%. FIG. 2 shows the biomass results for a strain of S. cerevisiae grown in 1× and 2×CMF in CMF-CMB media with ethanol addition.

### Example 4

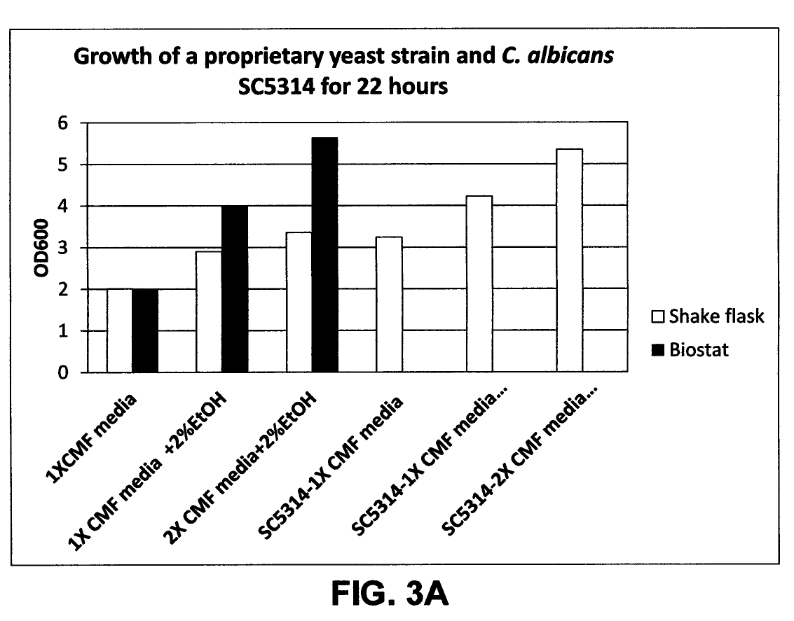

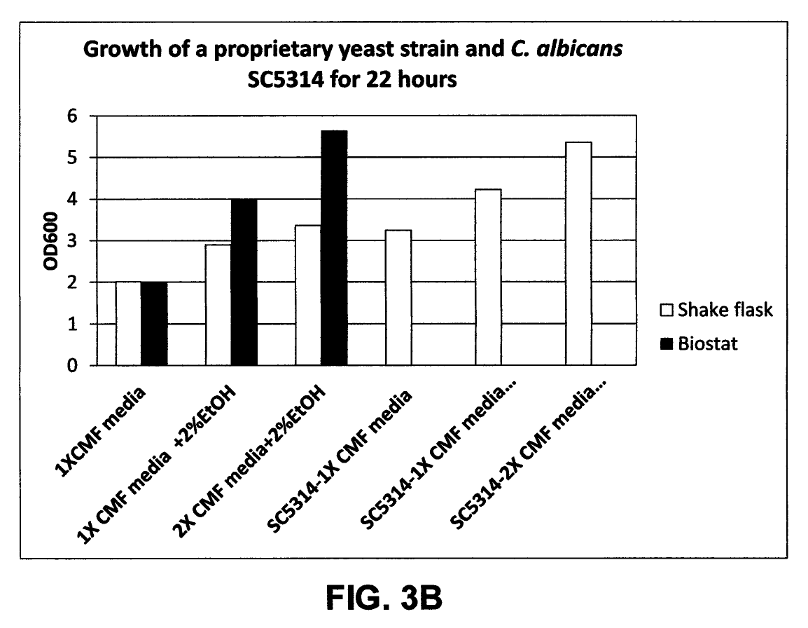

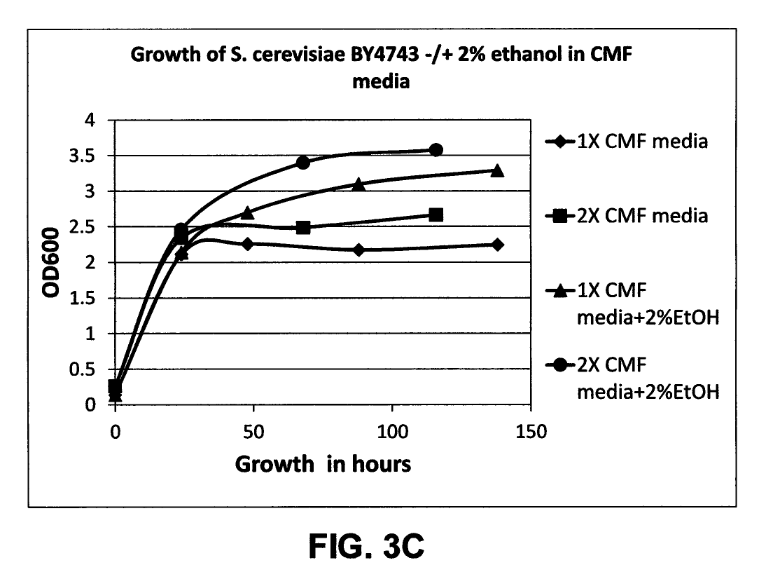

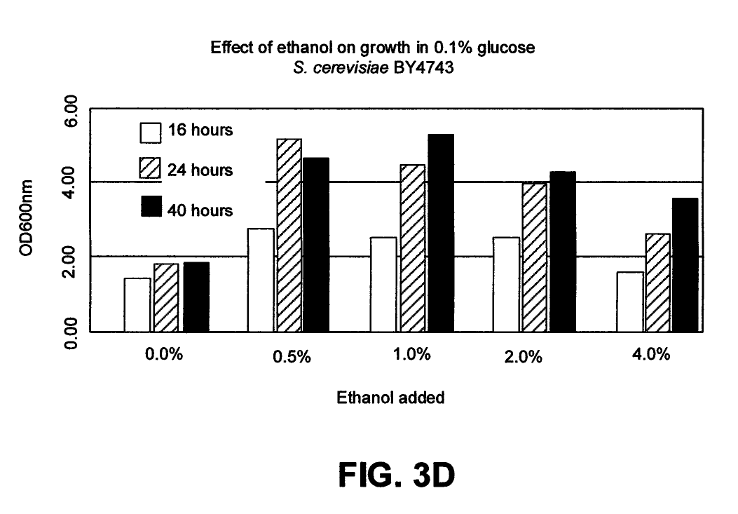

The benefit of ethanol addition to growth medium also applies to other yeast strains including, for example, S. cerevisiae BY4743 and W3031B, or to Candida albicans. FIGS. 3A, 3B, and 3C show the results of enhanced biomass production of various yeast strains with the addition of 2% ethanol during growth. A proprietary S. cerevisiae strain, Candida albicans SC5314 and S. cerevisiae strains BY4743 and W303-1B were grown in CMF-CMB alone or CMF-CMB media supplemented with amino acids. The cultures of the proprietary strain and C. albicans SC5314 were grown for 22 hours, and S. cerevisiae BY4743 and W3031B were grown for 144 hours. FIG. 3A shows the growth of the proprietary strain at 22 hours in both a shake flask and a Biostat fermentor with or without 2% ethanol and 1× or 2×CMF media. It also shows the growth of SC5314 −/+2% ethanol with 1× or 2×CMF media at 22 hours using the shake flask method. FIG. 3B and FIG. 3C represent growth curves of S. cerevisiae W3031B and BY4743, respectively, using the shake flask method and measured from 0 to 144 hours, −/+2% ethanol with 1× or 2×CMF media. As is evident from the data, ethanol addition significantly enhanced yeast growth in cultures.

### Example 5

In another example, Synthetic Complete Medium (“SCM”) has been used to grow yeast strains that have been genetically altered (“engineered”) to require certain amino acids and nitrogenous bases. Strains engineered by mutagenesis and genetic selection (e.g. S. cerevisiae W303-1B; Rothstein, R J Methods Enzymol. 1983; 101:202-11, PMID 6310324) were tested. This medium was also used to grow yeast strains engineered by homologous recombination with artificial DNA sequences designed to delete a specific gene (S. cerevisiae BY4743; Giaver, Nature, Vol. 418, 25 Jul. 2002). This same strain is also an example of yeast engineered by homologous recombination to add specific DNA sequences. Therefore, the following examples include yeast strains altered by genetic recombination to delete and to add specific DNA sequences.

YNB (Yeast Nitrogen Base) Medium, commercially obtained from Sunrise Biosciences

Salts, Vitamins, Minerals & Trace Elements:
Biotin (0.002 mg/L)
Boric acid (0.5 mg/L)
Calcium chloride dihydrate (100 mg/L)
Copper (II) sulfate pentahydrate (0.04 mg/L)
Folic acid (0.002 mg/L)
Inositol (In Base Formula) (2 mg/L)
Iron (III) chloride (0.2 mg/L)
Magnesium sulfate anhydrous (500 mg/L)
Manganese sulfate monohydrate (0.4 mg/L)
Niacin (0.4 mg/L)
4-Aminobenzoic acid (PABA) (In Base Formula) (0.2 mg/L)
D-Pantothenic acid hemicalcium salt (0.4 mg/L)
Potassium iodide (0.1 mg/L)
Potassium phosphate monobasic anhydrous (1000 mg/L)
Pyridoxine hydrochloride (0.4 mg/L)
Riboflavin (0.2 mg/L)
Sodium chloride (100 mg/L)
Sodium molybdate (0.2 mg/L)
Thiamine hydrochloride (0.4 mg/L)
Zinc sulfate monohydrate (0.4 mg/L)
Ammonium sulfate (4.5 g/L)

CSM Amino Acids & Supplements (Sunrise Biosciences)
Adenine hemisulfate (10 mg/L)
L-Arginine (50 mg/L)
L-Aspartic acid (80 mg/L)
L-Histidine hydrochloride monohydrate (20 mg/L)
L-Isoleucine (50 mg/L)
L-Leucine (100 mg/L)
L-Lysine hydrochloride (50 mg/L)
L-Methionine (20 mg/L)
L-Phenylalanine (50 mg/L)
L-Threonine (100 mg/L)
L-Tryptophan (50 mg/L)
L-Tyrosine (50 mg/L)
L-Valine (140 mg/L)
Uracil (20 mg/L)

To this medium, fermentable and non-fermentable Carbon sources are added.

In this example, S. cerevisiae BY4743 was grown in this medium to test optimal concentrations of fermentable and non-fermentable C sources for this strain. Identical 75 μL inocula in Synthetic Complete Medium with 0.1% (1 g/L) glucose were added to 25 mL of the same medium in separate cultures with various amounts of glucose and incubated at 30° C. Glucose concentration was varied between 0.05% (0.5 g/L) and 0.5% (5 g/L). After 5 hours, ethanol was added in amounts between 0 and 4 volume percent. Growth and biomass were monitored as OD_{600 nm} for 40 hours. Optimal growth rate and maximal biomass at 24 hours resulted from growth in the presence of 0.1% glucose results after addition of 0.5 volume percent ethanol (FIG. 3D), and the addition of ethanol under these conditions doubles biomass. A similar analysis shows that for this strain, 0.25% glucose is optimal for biomass production in the presence of 2% ethanol.

### Example 6

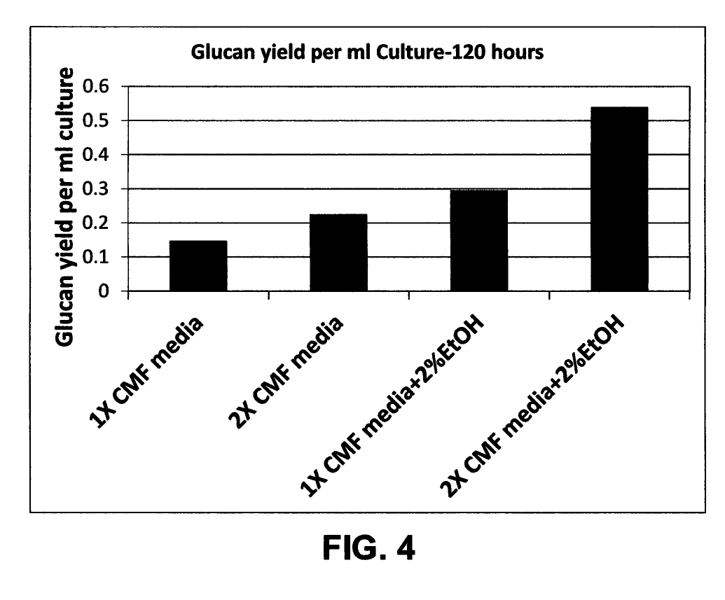

As shown in FIG. 4, growth in ethanol also increased the yield of a commercially marketed yeast natural product, β-glucan obtained from the biomass. The increased yield was due to increased biomass as well as increased yield of β-glucan per gram of biomass. A starter culture is grown in CMF-CMB media overnight. The starter inoculum was added to respective media as labeled in FIG. 4 to achieve a uniform starting OD. The cultures are incubated in a shaker incubator at 30° C. and 170 rpm for 4 to 5 hour or about 2 generations and then 2% ethanol is added. The cultures are incubated back at 30° C. and OD₆₀₀ was measured at 120 hours and the cultures are harvested. The culture pellets were alkali extracted for glucan and lyophilized. Dry mass of extracted glucans is measured. The glucan samples are subjected to GEM analysis for glucan content. Glucan yield per OD is calculated as (GEM glucan %*dry weight of extracted glucan)/(100*OD600 at 120 hours).

### Example 7

In another example, yeast mutants are engineered to improve yield of a desired natural product, namely yeast β-glucan. From a set of mutants with specific genes deleted and “barcoded” by homologous recombination (Giaver, Nature, Vol. 418, 25 Jul. 2002), we selected a panel of 200 genes known to affect cell wall structure or composition, and screened them for production of the desired product, β-glucan. These mutants are grown in grown in CMB medium supplemented with CSM Amino Acids and Supplements using 1×CMF as fermentable carbon source. Of the tested mutants, a deletion of gene VRP1 leads to a 50% increase in β-glucan compared to the parental strain BY4743). This gene is among those screened due to its previous characterization as a wall-related mutant (Donnelly DOI: 10.1111/j.1365-2958.1993.tb00930.x). Growth of this mutant in the same medium supplemented with 2 volume percent ethanol leads to a doubling of β-glucan content compared to the parental strain. Therefore, growth of a genetically altered yeast strain in medium with limited fermentable carbon supplemented with 2% ethanol significantly increases the yield of a desired product of yeast such as β-glucan.

In summary, growth of yeasts in media containing concentrations of fermentable C sources at 0.5% or below, with ethanol increases growth rate, prolongs growth, increases biomass produced, and increases yield of specific products of yeast growth. This enhancement is seen in multiple yeast strains, species, and growth media. This finding promises to increase efficiency of industrial and research processes dependent on growing yeast, including, but not limited to, natural yeast products, yeast product foodstuffs, and biopharmaceuticals expressed by genetic modifications in yeast.

The complete disclosure of all patents, patent applications, and publications, and electronically available material cited herein are incorporated by reference in their entirety. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

What is claimed is:

1. A process for increasing yeast biomass, the process comprising:
culturing yeast cells in a growth medium comprising a fermentable carbon source, wherein the yeast cells grow at a growth rate;
adding ethanol before the growth rate begins to decrease; and
permitting the yeast cells to continue to grow after the step of adding ethanol.

2. The process as recited in claim 1, wherein the ethanol has a concentration of between 0.5% and 4.0 volume percent in the growth medium.

3. The process as recited in claim 1, wherein the ethanol has a concentration of between 1.0 volume percent and 3.0 volume percent in the growth medium.

4. The process as recited in claim 1, wherein the fermentable carbon source is between 0.05 mass percent and 0.5 mass percent in the growth medium.

5. The process as recited in claim 1, wherein the culturing, the adding and the permitting all occur at a temperature between 24° C. and 40° C.

6. A process for increasing yeast biomass, the process comprising sequentially:
adding yeast cells to a growth medium comprising a fermentable carbon source at a concentration of between 0.05 mass percent and 0.5 mass percent in the growth medium;
culturing the yeast cells in the growth medium for a least one hour;
adding ethanol, after the at least one hour, to the growth medium such that the ethanol has a concentration of between 0.5 volume percent and 4.0 volume percent, wherein the adding ethanol occurs within ten hours of the adding yeast cells; and
permitting the yeast cells to continue to grow after the step of adding ethanol.

7. The process as recited in claim 6, wherein the step of adding ethanol adds a composition of matter that consists only of aqueous ethanol.
